(19) United States
(12) Patent Application Publication
(10) Pub. No.: US 2008/0033005 A1
(43) Pub. Date: Feb. 7, 2008

(54) METHODS FOR THE TREATMENT OF MULTIPLE SCLEROSIS

(75) Inventors: Jean E. MERRILL, Morristown, NJ (US); Sandrines FUNES, Nogent-sur-marne (FR); Wayne PETKO, South Bound Brook, NJ (US); Friederike WIRTZ-BRUGGER, Branchburg, NJ (US); Karen CHANDROSS, Somerset, NJ (US)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: AVENTIS PHARMACEUTICALS INC., Bridgewater (NJ)

(21) Appl. No.: 11/764,259

(22) Filed: Jun. 18, 2007

Related U.S. Application Data
(63) Continuation of application No. PCT/US05/45295, filed on Dec. 14, 2005.
(60) Provisional application No. 60/640,926, filed on Dec. 31, 2004.

(51) Int. Cl. A61K 31/4375

(52) U.S. Cl. 514/284

(57) ABSTRACT

Certain compounds, including 5,6-dihydro-3,9-dihydroxyindolo[2,1-a-isoquinolin12yl)[4-[2-(1-piperidinyl)ethoxy]phenyl]-methanone and arzoxifen, are useful for providing protection to oligodendrocytes and neurons of multiple sclerosis patients.

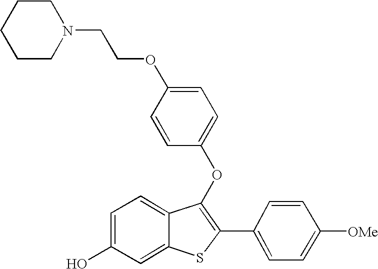

### CROSS-REFERENCE TO RELATED APPLICATIONS

This application is a continuation of International Patent Application No. PCT/US2005/045295 filed on Dec. 14, 2005 which is incorporated herein by reference in its entirety which also claims the benefit of priority of U.S. Provisional Patent Application No. 60/640,926 filed on Dec. 31, 2004.

### FIELD OF THE INVENTION

The present invention relates to methods of treating multiple sclerosis. In particular, the present invention relates to the treatment of multiple sclerosis through the protection of the central nervous system on a cellular level through the inhibition of demyelination of neurons and/or oligodendrocytes afflicted with the disease with certain compounds disclosed herein, their isomers, racemates, enantiomers, their salts, and medicaments containing them.

### BACKGROUND OF THE INVENTION

Multiple sclerosis (MS) is an autoimmune disease that leads to a loss of CNS (central nervous system) myelin, oligodendrocyte cell death and axonal destruction, causing severe functional deficits. MS occurs at a 2-3 times higher incidence in women than men (Duquette, et al., 1992. Can. J. Neurol. Sci. 19: 466-71.) and estrogen reduces disease severity during the second and third trimesters of pregnancy (Confavreux et al., 1998. N Eng J Med 339: 285-291), whereas the clinical symptoms of MS have been reported to exacerbate after delivery (Evron et al., 1984. Am. J. Reprod. Immunol. 5:109-113; Mertin and Rumjanek 1985. J. Neurol Sci. 68:15-24; Grossman, 1989. J. Steroid Biochem. 34: 241-245; Confavreux et al., 1998. N. Engl. J. Med. 339: 285-291). Treatment with estradiol decreases gadolinium-enhancing lesions and MRI volume (Voskuhl and Palaszynski, 2001. Neuroscientist. 7(3): 258-270; Sicotte et al., 2002. Ann Neurol. 52: 421-428). Furthermore, estrogens cause immune response shifts, amelioration of clinical symptoms and enhanced myelin formation in rodent EAE (experimental allergic encephalomyelitis) (Curry and Heim 1966. Nature 81: 1263-1272; Kim et al., 1999. Neurology. 52: 1230-1238; Ito et al., 2002. Clin Immunol. 102(3): 275-282). Estrogen has been reported to protect oligodendrocytes from cytotoxicity induced cell death (Takao et al, 2004. J Neurochem. 89: 660-673) and 17β-estradiol (E2) has been reported to hasten the elaboration of multiple, interconnecting processes on oligodendrocytes (Zhang et al., 2004. J Neurochem 89: 674-684).

There is increasing evidence that estrogen plays a direct protective role in response to degenerative disease and injury by enhancing cell survival, axonal sprouting, regenerative responses, synaptic transmission, and neurogenesis. In the CNS, there is increased synthesis of estrogen and enhanced expression of the estrogen receptors at sites of injury (Garcia-Segura et al., 2001. Prog. in Neurobiol. 63: 29-60.) and estrogen-mediated cellular protection has been demonstrated in a number of in vitro models of neurodegeneration, including β-amyloid induced cytotoxic, excitotoxicity, and oxidative stress (Behl et al., 1995. Biochem. Biophys. Res. Commun. 216,473-482; Goodman et al., 1996. J. Neurochem. 66:1836-1844; Green et al., 1997. J. Neurosci. 17: 511-515; Behl et al., 1999. Trends Pharmacol. Sci. 20: 441-444). Recent clinical studies suggest that estrogen replacement therapy may also decrease the risk and delay the onset and progression of Alzheimer's disease and schizophrenia. (For a review see Garcia-Segura et al., 2001. Prog. in Neurobiol. 63: 29-60.) E2, a lipophilic hormone that can cross the blood-brain barrier, and metabolically sustain and maintain those sections of the brain responsible for arousal, attention, mood, and cognition (Lee and McEwan, 2001. Annu. Rev. Pharmacol. & Toxicol. 41: 569-591.). In addition, both natural estrogens and synthetic selective estrogen receptor modulators (SERMs), such as tamoxifen, decrease neuronal damage caused by ischemic stroke, while either E2 or raloxifene protect neurons against 1-methly-4-phenyl-1,2,3,6 tetrahydropyridine-induced toxicity (Callier, et al., 2001. Synapse 41: 131-138; Dhandapani and Brann, 2003. Endocrine 21: 59-66).

The neuroprotective effects of estrogen are mediated through the modulation of bcl-2 expression, the activation of cAMP and mitogen-activated kinase signaling pathways, the modulation of intracellular calcium homeostasis, the enhancement of antioxidant activity, and/or activation of estrogen receptors (ER) that can act as hormone-regulated transcription factors (Mangelsdorf, et al., 1995. Cell 83: 835-839; Katzenellenbogen, et al., 1996. Mol. Endocrinol. 10: 119-131; Singer et al., 1996. Neurosci. Lett. 212: 13-16; Singer et al., 1998. Neuroreport 9: 2565-2568; Singer et al., 1999. Neurosci. Left. 212: 13-16; Weaver et al., 1997. . Brain Res. 761: 338-341; Watters and Dorsa, 1998. J. Neurosci. 18: 6672-6680; Singh et al., 1999. J. Neurosci. 19: 1179-1188; Alkayed et al., 2001. J. Neurosci. 21: 7543-7550; Garcia-Segura et al., 2001. Prog. in Neurobiol. 63: 29-60). Two estrogen receptors that have been characterized, ERα and ERβ, belong to the class I hormone receptor family that function as nuclear transcription factors. ERα and ERβ (in the form of mRNA or protein) are expressed in neural cell types including Schwann cells, the myelin forming cells of the peripheral nervous system, and CNS neurons, astrocytes and oligodendrocytes (Miranda and Toran-Allerand, 1992; Santagati, et al., 1994; Kuiper, et al., 1996; Mosselman, et al., 1996; Thi et al. 1998; Platania, et al., 2003). In oligodendrocytes, the myelin forming cells of the CNS that are lost in MS, ERα has been reported to be nuclear, whereas ERβ is cytoplasmic, in vivo immunoreactivity being readily detectable in the cytoplasm and myelin sheaths (Zhang et al., 2004. J Neurochem 89: 674-684). Recently, Arvanitis at al., 2004 (J Neurosci Res. 75: 603-613) have reported an ER with similarities to ERβ in isolated CNS myelin, the myelin sheath of spinal cord and brain sections and the oligodendrocyte plasma membrane.

Mimicking and/or enhancing the beneficial effects of estrogen in MS by means of small molecules that are ligands at ERA, or compounds that preferentially mimic the effects of estrogen at sites other than the classical ERα is likely to have advantages for the treatment of MS in that the small molecules would be devoid of the untoward “hormonal” effects of estrogen which are mediated by ERα. These other ER sites may include the recently identified ER-X, which has been identified in neurons and is developmentally regulated (Toran-Allerand 2004. Endocrinology 145:1069-1074), or GPR30, which allows estrogen to trigger different pathways that integrate cell surface signaling with gene transcription (Kanda and Watanabe 2003. J Invest Derm 121: 771-780).

These compounds may also be used to treat or prevent the development of other demyelinating diseases, including Charcot-Marie-Tooth disease, Pelizaeus-Merzbacher disease, encephalomyelitis, neuromyelitis optica, adrenoleukodystrophy, Guillain-Barre syndrome, and disorders in which myelin-forming glial cells (oligodendrocytes or Schwann cells) are damaged, including spinal cord injury, neuropathies and nerve injury.

### SUMMARY OF THE INVENTION

Certain compounds, including 5,6-dihydro-3,9-dihydroxyindolo[2,1-a-isoquinolin-1,2-yl)[4-[2-(1-piperidinyl)ethoxy]phenyl]-ethanone and arzoxifen, are useful for the metabolic protection and maintenance of oligodendrocytes and neurons of multiple sclerosis patients.

### DETAILED DESCRIPTION OF THE INVENTION

The present invention comprises the treatment of multiple sclerosis through the protection of the central nervous system on a cellular level through the inhibition of de-myelination of neurons and/or oligodendrocytes afflicted with the disease. This therapeutic endeavor is achieved through the administration of one of two compounds, 5,6-dihydro-3,9-dihydroxyindolo[2,1-a-isoquinolin-1,2-yl)[4-[2-(1-piperidinyl)ethoxy]phenyl]-methanone and arzoxifen, their acid-addition salts of inorganic or organic acids and formulations comprising them.

These compounds, 5,6-dihydro-3,9-dihydroxyindolo[2,1-a-isoquinolin-1,2-yl)[4-[2-(1-piperidinyl)ethoxy]phenyl]-methanone and arzoxifen, contain one or more asymmetric centers and thereby have isomeric forms and these isomers and mixtures thereof also fall within the penumbra of the claimed invention as well as the racemates and the enantiomers thereof. Structurally, the compounds useful in the claimed methods of treatment are as follows:

A) 5,6-dihydro-3,9-dihydroxyindolo[2,1-a]isoquinolin-12-yl)[4-[2-(1-piperidinyl)ethoxy]phenyl]-methanone

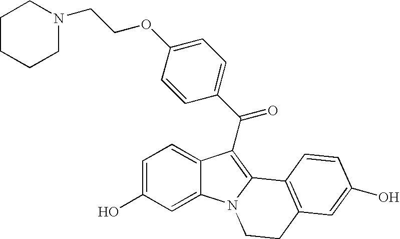

B) Arzoxifen

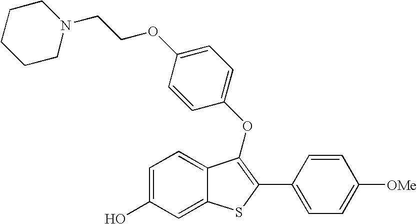

The compounds may be formulated as described herein below with excipients, diluents, solvents, binders, lubricants, carriers and other traditional pharmaceutically acceptable components known in the art.

As set forth below, the terms used in this specification have their respective meanings defined as follows.
a) “Pharmaceutically acceptable salts” means either an acid addition salt or a basic addition salt, whichever is necessary possible to make with the compounds of the present invention.
“Pharmaceutically acceptable acid addition salt” is any non-toxic organic or inorganic acid addition salt of the base compounds represented by Formula I. Illustrative inorganic acids which form suitable salts include hydrochloric, hydrobromic, sulfuric and phosphoric acid and acid metal salts such as sodium monohydrogen orthophosphate and potassium hydrogen sulfate. Illustrative organic acids which form suitable salts include the mono-, di- and tri-carboxylic acids. Illustrative of such acids are, for example, acetic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, salicylic, 2-phenoxybenzoic, p-toluenesulfonic acid and sulfonic acids such as methanesulfonic acid and 2-hydroxyethanesulfonic acid. Either the mono- or di-acid salts can be formed, and such salts can exist in either a hydrated or substantially anhydrous form. In general, the acid addition salts of these compounds are more soluble in water and various hydrophilic organic solvents and which in comparison to their free base forms, generally demonstrate higher melting points.
“Pharmaceutically acceptable basic addition salts” means non-toxic organic or inorganic basic addition salts of the compounds of Formula I. Examples are alkali metal or alkaline-earth metal hydroxides such as sodium, potassium, calcium, magnesium or barium hydroxides; ammonia, and aliphatic, alicyclic, or aromatic organic amines such as methylamine, trimethylamine and picoline.

The selection of the appropriate salt may be important so that the ester is not hydrolyzed. The selection criteria for the appropriate salt will be known to one skilled in the art.
b) “Patient” means a warm blooded animal, such as for example rat, mice, dogs, cats, guinea pigs, and primates such as humans.
c) “Treat” or “treating” means any treatment, including, but not limited to, alleviating symptoms, eliminating the causation of the symptoms either on a temporary or permanent basis, or preventing or slowing the appearance of symptoms and progression of the named disorder or condition.
d) “Therapeutically effective amount” means an amount of the compound, which is effective in treating the named disorder or condition.
e) “Pharmaceutically acceptable carrier” is a non-toxic solvent, dispersant, excipient, adjuvant or other material which is mixed with the compound of the present invention in order to permit the formation of a pharmaceutical composition, i.e., a dosage form capable of administration to the patient. One example of such a carrier is pharmaceutically acceptable oil typically used for parenteral administration.
f) “Stereoisomers” is a general term for all isomers of the individual molecules that differ only in the orientation of their atoms in space. It includes mirror image isomers (enantiomers), geometric (cis/trans) isomers, and isomers of compounds with more than one chiral center that are not mirror images of one another.

In treating a patient afflicted with a condition described above, a selected compound can be administered in any form or mode which makes the compound bioavailable in therapeutically effective amounts, including orally, sublingually, buccally, subcutaneously, intramuscularly, intravenously, transdermally, intranasally, rectally, topically, and the like. One skilled in the art of preparing formulations can determine the proper form and mode of administration depending upon the particular characteristics of the compound selected for the condition or disease to be treated, the stage of the disease, the condition of the patient and other relevant circumstances. For example, see Remington's Pharmaceutical Sciences, 18th Edition, Mack Publishing Co. (1990), incorporated herein by reference.

The compositions of the present invention may be administered orally, for example, in the form of tablets, troches, capsules, elixirs, suspensions, solutions, syrups, wafers, chewing gums and the like and may contain one or more of the following adjuvants: binders such as microcrystalline cellulose, gum tragacanth or gelatin; excipients such as starch or lactose, disintegrating agents such as alginic acid, Primogel, corn starch and the like; lubricants such as magnesium stearate or Sterotex; glidants such as colloidal silicon dioxide; and sweetening agents such as sucrose or saccharin may be added or a flavoring agent such as peppermint, methyl salicylate or orange flavoring. When the dosage unit form is a capsule, it may contain, in addition to materials of the above type, a liquid carrier such as polyethylene glycol or a fatty oil. Other dosage unit forms may contain other various materials, which modify the physical form of the dosage unit, for example, as coatings. Thus, tablets or pills may be coated with sugar, shellac, or other enteric coating agents. A syrup may contain, in addition to the present compounds, sucrose as a sweetening agent and certain preservatives, dyes and colorings and flavors.

The compounds of this invention may also be administered topically, and when done so the carrier may suitably comprise a solution, ointment or gel base. The base, for example, may comprise one or more of petrolatum, lanolin, polyethylene glycols, bee wax, mineral oil, diluents such as water and alcohol, and emulsifiers and stabilizers.

The solutions or suspensions may also include one or more of the following adjuvants: sterile diluents such as water for injection, saline solution, fixed oils, polyethylene glycols, glycerine, propylene glycol or other synthetic solvents; antibacterial agents such as benzyl alcohol or methyl paraben; antioxidants such as ascorbic acid or sodium bisulfite; chelating agents such as ethylene diaminetetraacetic acid; buffers such as acetates, citrates or phosphates and agents for the adjustment of tonicity such as sodium chloride or dextrose. The parenteral preparation can be enclosed in ampules, disposable syringes or multiple dose vials.

The dosage range at which compounds of the invention exhibit their ability to act therapeutically can vary depending upon the particular compound, the severity of the condition, the patient, the formulation, other underlying disease states that the patient is suffering from, and other medications that may be concurrently administered to the patient. Generally, the compound of Formula I will exhibit their therapeutic activities at dosages of between about 0.001 mg/kg of patient body weight/day to about 100 mg/kg of patient body weight/day.

The contents of all publications and patents discussed herein are hereby incorporated herein by reference.

It will be appreciated that every suitable combination of the respective elements of the present invention may be interchanged with one or more of other similar, suitable components known in the art and changed in minor, non-functional respects. These additional embodiments of the invention are also regarded as falling within the scope of the claims herein. The examples detailed below are provided to better describe and more specifically set forth the elements and mechanics/operation of the present invention with reference to the drawing, but for obvious reasons cannot describe all of them. It is to be recognized that the examples therefore are for illustrative purposes only however, and should not be interpreted as limiting the spirit and scope of the invention as later recited by the claims that follow.

Neuroprotection Assay

Cells from a human neuroblastoma cell line, SK-N-SH cells, were plated at 50,000 cells/well in Costar Biocoat 96-well poly-D-lysine coated plates in EMEM (Minimum Essential Medium Eagle with Earle's salts) containing penicillin/streptomycin, L-glutamine, sodium pyruvate, non-essential amino acids and sodium bicarbonate. Cells were grown overnight in a 37° C. incubator under 5% CO₂. The next day, the medium was removed and replaced with fresh medium. Cells were pretreated with Serms for 1 hour, and SIN-1 (3-morpholinosydnonimine, which produces peroxynitrite) was added to give a final concentration of 2 or 10 mM. After 24 hours, the medium was removed and assayed for LDH activity using the Promega cytotox 96 kit (catalog# G1780). Results were calculated as percent protection against SIN-1 toxicity.

ERK1/2 Westerns

SK-N-SH cells were plated at 2×10⁶ cells/well in 6-well polystyrene culture plates, in 2 ml EMEM containing penicillin/streptomycin, L-glutamine, sodium pyruvate, non-essential amino acids and sodium bicarbonate. Cells were grown overnight at 37° C. under 5% CO₂.

The next day, 200 μl medium was removed and cells were dosed with 200 μl compound made up to 10 times the final concentration in medium. After incubation for the appropriate time, medium was aspirated off and cells washed twice with cold PBS. They were then lysed with 100 μl RIPA buffer containing protease and phosphatase inhibitors.

For westerns, 20 μg protein was denatured at 95° C. in Laemmli sample buffer containing beta-mercaptoethanol, then loaded onto 4-20% gradient Tris Glycine SDS gels and electrophoresed at 70 volts until completed. Proteins were transferred to nitrocellulose membranes and probed for phospho-ERK1/2 and total ERK1/2 using the appropriate antibodies. Bands were detected using ECL western blotting chemiluminescent substrate. For phospho-ERK ELISA's, the ELISA kit from Assay Designs was used.

Bcl-2 Luciferase

SK-N-MC Bcl-2 (neo) clone 218 was plated at 25,000 cells per well in Packard View plates in phenol Red free EMEM containing penicillin/streptomycin, L-glutamine, sodium pyruvate, non-essential amino acids, sodium bicarbonate and 200 ug/ml G418. Cells were grown overnight in a 37° C. incubator under 5% CO2.

On day 2, medium was removed and replaced with serum-free EMEM containing ITS supplement (BD Biosciences # 35 4352). Medium was changed again on days 3 and 4; on day 4 cells were dosed with compounds, in a final volume of 100 μl. Twenty-four hours after dosing, 100 μl SteadyGlo (Promega# E2510) was added and luciferase measured in a Packard Topcount liquid scintillation counter.

Oligodendrocyte Toxicity Assay

Primary rat oligodendrocyte progenitor cells were obtained from the cerebra of 2-3 day old postnatal rats (Sprague Dawley). The meninges were removed and tissue was mechanically dissociated. Cells were plated on T75 flasks and fed with DMEM+10% FBS.

Enriched OLPs were collected by mechanical separation from the astrocytic monolayer and were expanded in serum free media (SFM) supplemented with the mitogens, PDGF-AA (10 ng/ml) and FGF-2 (10 ng/ml).

To generate mature oligodendrocytes, progenitor cells were switched to SFM supplemented with IGF-1 (10 ng/ml) 24 hours after plating and cells were grown under these conditions for 7 days prior to experimental assays.

Cells were plated in 96-well plates, 10,000 per well. Medium was changed to fresh medium and cells were pretreated with compounds for 1 hour. Toxins were added to give the following final concentrations:
Sin-1 10 mM
Pyrogallol 500 μM
C2 ceramide 100 μM
Camptothecin 10 μM

After 24 hours, medium was removed and assayed for LDH activity using the Promega cytotox 96 kit (catalog# G1780). Results were calculated as percent protection against toxin-induced toxicity.

These compounds have been assessed for their efficacy in neuroprotection against cell death produced by toxic agents such as SIN-1 (3-morpholino-sydnonimine, producing peroxynitrite), C2 ceramide, camptothecin, staurosporine, SNAP (S-nitroso-N-acetylpenicillamine, producing nitric oxide), and pyrogallol (producing superoxide anion). The target cells assessed in vitro are: human neuroblastoma cell lines [SK-N-SH, SH-SY5Y], and primary cultures of rodent oligodendrocyte progenitors and their mature counterparts. Protection by these serm-like compounds have been compared to 17-β-estradiol and tamoxifene. (See Table 1 below) The mechanism of action of this neuroprotection has been investigated with respect to the use of a classical nuclear (genomic) ERα or β and an assessment of the role for phosphorylation of MAPK p40/p42 (ERK1/2).

### RESULTS

Pharmacological Composition

| | | ER□ selctivity | | % Protection | |
|---|---|---|---|---|---|
| | | ER□ | ER□ | (SIN-1*) | |
| | | Binding | binding | OLP | OL |
| Cpd | Structure | IC50 nM | IC50 nM | 10 nm | 10 nm |
| Arzoxifen | 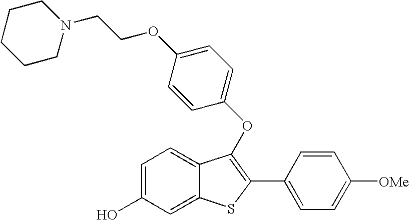 | 69.8 | 64.5 | 1.485 | 8.237 |
| (5,6-dihydro- 3,9- dihydroxyindolo [2,1- a]isoquinolin- 12-yl) [4-[2-(1- piperidinyl) ethoxy]phenyl]- methanone | 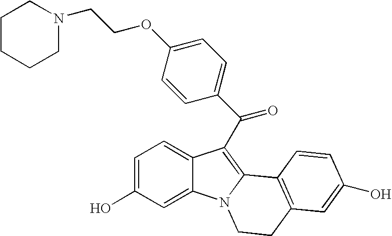 | >1000 | >1000 | 6.049 | >30 |

Both compounds tested appear to protect neurons and oligodendrocytes. This appears to be mediated by the upregulation of ERK1/2 phosphorylation, which is confirmed by inhibition of the neuroprotection by U-0126, a MEK inhibitor, specific for the ERK pathway.

What is claimed is:

1. A method for the treatment of multiple sclerosis which comprises administering to a patient in need thereof a therapeutically effective amount of a compound selected from the group consisting of 5,6-dihydro-3,9-dihydroxyindolo[2,1-a-isoquinolin12yl)[4-[2-(1-piperidinyl)ethoxy]phenyl]-methanone and arzoxifen, their isomers, racemates enantiomers, and the pharmaceutically acceptable salts thereof.

2. The method of claim 1 wherein said effective amount is administered daily and is in the range from about 0.001 to about 100 mg/kg of patient body wt./day.

3. The method of claim 2 wherein said compound is formulated in a pharmaceutically acceptable carrier composition suitable for administration to the patient orally, sublingually, buccally, subcutaneously, intramuscularly, intravenously, transdermally, intranasally, rectally and topically.

4. The method of claim 3 wherein said composition is formulated as a tablet, troche, capsule, elixir, suspension, solution, syrup, wafer, or chewing gum

5. The method of claim 4 wherein said composition comprises adjuvants, binders, disintegrating agents lubricants excipients; lubricants, glidants, solvents, diluents, flavoring agents and sweetening agents.

6. The method of claim 5 wherein said adjuvants are selected from the group consisting of microcrystalline cellulose, gum tragacanth, gum Arabic, carageenan, gelatin and mixtures thereof.

7. The method of claim 5 wherein said excipients are selected from the group consisting of starch, corn starch, lactose, cellulose and cellulose derivatives, sugars, magnesium stearate, colloidal silicon dioxide, vegetable stearine, alginic acid and mixtures thereof.

8. The method of claim 6 wherein said sweetening agents are selected from the group consisting of sucrose, saccharin, aspartame, acesulfame-K, sucralose and mixtures thereof.

9. The method of claim 8 wherein said solvent further comprises a liquid carrier selected from the group consisting of polyethylene glycol, water, a sugar alcohol, a fatty alcohol, mineral oil, vegetable oil and mixtures thereof
